(19) United States
(12) Patent Application Publication
(10) Pub. No.: US 2008/0280777 A1
(43) Pub. Date: Nov. 13, 2008

(54) Method for determining the effects of external stimuli on biological pathways in living cells

(75) Inventors: Michael Bittner, Phoenix, AZ (US); Edward R. Dougherty, College Station, TX (US)

Correspondence Address: DLA PIPER US LLP, 4365 EXECUTIVE DRIVE, SUITE 1100, SAN DIEGO, CA, 92121-2133, US

(73) Assignee: Translational Genomics Research Institute, Phoenix, AZ (US)

(21) Appl. No.: 12/118,352

(22) Filed: May 9, 2008

Related U.S. Application Data
(60) Provisional application No. 60/928,816, filed on May 11, 2007.

(51) Int. Cl. C40B 30/04; C12Q 1/68; G06K 9/46; G06G 7/48

(52) U.S. Cl. 506/9; 435/6; 703/11; 382/133

(57) ABSTRACT

The present invention describes methods for carrying out experiments on living cells, including making measurements of the operation transcriptional regulatory processes and indicators of the kinds of processes operating in the cell in response to external stimuli. Image analysis allows for gathering data concerning the flow of information through a cell's genomic regulatory network as it is executing a programmatic change in its activities as a function of said stimuli. The method also allows collection of data of the results of the information-processing in the cell by observing the decisions the cell makes when modulating cellular process activities.

### CROSS REFERENCE TO RELATED APPLICATION

This application claims priority under 35 U.S.C. §119(e) to U.S. Provisional Application No. 60/928,816, filed on May 11, 2007, which is incorporated herein by reference in its entirety.

### BACKGROUND OF THE INVENTION

1. Field of the Invention

The present invention relates generally to systems biology, and more specifically to in situ methods of determining the effects of external stimuli on cell signaling circuitry.

2. Background Information

Biological systems like any other complex system, rely on the functioning of many components that are organized into sub-systems, each of which carries out particular process that is required for the functioning of the complete system. In multicellular organisms, all of the cells use a very similar set of core sub-systems that are required for maintenance of each cell's integrity and basic functionality. These subsystems differ mainly in their levels of activity, which is dependent on what specialized functions a particular cell type must carry out. These core processes include functions that allow the cell to adaptively respond to stress and damage.

Multicellular organisms typically develop from a single cell. During development from that cell, the growing masses of cells interact with each other and the environment to produce a body where the cells in particular organs carry out types of specialized activities that are specific to the type of tissue (e.g., heart, lung, brain . . . etc.) they are associated with. These specialized activities may be carried out continuously or sporadically. Cells that have to support these mixes of common and tissue specific activities have to be very adroit at regulating the sets of sub-systems that are active at any given time, and at using the available information about internal and external conditions to decide which alterations in the kinds of activities of the sub-systems are required. Again, there are striking similarities between complex manmade systems and biologic ones. Just as manmade devices for a wide variety of purposes can be made with a fairly limited number of elementary, interacting constituents that are combined to function in alternative ways, cells create a wide variety of functions simply by altering the combinations of interacting constituents present in the cell.

In essence, all fields of biology where it would be useful to be able to modulate cellular systems to alter their function in ways that would enable or enhance subsystem processing, allow the systems to operate under different environmental conditions or reduce disease or parasitism would benefit from biological analogs of the tools available to engineers that design, characterize and control complex man-made systems.

The study of the complex interactions between cellular components that allow the cell to carry out the myriad interrelated functions necessary for life is often referred to as “systems biology.” This is a very broad term, covering very diverse topics and research goals. One way of subdividing the field is to consider the level in the cellular system that is being studied, the requirements in terms of data for studying that level, and the kinds of goals typical of such studies.

Systems biology approaches have been used to study metabolism. Such approaches require very detailed knowledge about genes specifying catalytic enzymes, the sequences of these genes, the biochemical characterizations of the small molecule inputs and outputs of the catalytic reaction, the known rates of conversion and typical metabolite levels and the knowledge of regulatory or intracellular localizing interactions that have already been acquired to produce a framework within which to examine metabolism in organisms where this level of knowledge has not yet been developed. Using such information it is possible to model the probable metabolic network for an organism with a sequenced genome, extrapolating the functions and relationships of other organism's metabolic network onto the new organism. While an extremely effective form of analysis, the requirement for so much detailed information of such different types is a strong limitation on the use of this kind of approach. No other components of the cell's network of comparable size have been characterized to this extent, which constrains this approach to the study of questions involving metabolism.

A second area of study that has less demanding requirements for the variety of data types required and the quantitative accuracy of the measurements is the area of development of multicellular organisms from their single-cell origin as an egg. As with metabolism, this area of study has the advantages of being a stepwise process, with identifiable intermediates and a standard progression. Depending on the complexity of the organism being studied, there can be very little to quite considerable redundancy in terms of the functions required to carry out each particular step, and genes can have differing roles in differing tissues, making this a much more challenging analysis. At the current stage, most of this work is focused on the very large challenge of simply understanding what molecular processes are involved in specifying the processes of delineation, spatial localization and acquisition of specialized features of the various tissues and body parts.

Other approaches are centered on the control of expression of genes, looking at the sequence elements in the promoter of each gene, and the transcription factors that interact with these elements to allow or prohibit gene expression. Enlarging on work that demonstrated the logical switch-like properties of gene promoters, many groups have carried out extensive experimentation on how the modification of various elements of the promoter alters the transcriptional properties of specific genes, elegantly describing how the series of these elements allow transcriptional factors present at specific times and locations in the embryo to specify particular transcriptional programs in genes that carry these elements. As in the case of metabolism, this kind of systems study involves a huge infrastructure of experimentation to derive the knowledge of the promoter elements and the transcription factors that interact with them. Once this knowledge is available, models that are predictive of how a specific perturbation on the network would affect the function of the network can be made. The accuracy of such predictions will be heavily influenced by how complete the information is with regard to overlap and redundancy of function and other compensatory mechanisms at work in the network.

### SUMMARY OF THE INVENTION

The present invention describes a method for carrying out experiments on living cells, making measurements of the transcriptional regulatory processes and analyzing the data produced. The method is useful for gathering data on the flow of information through a cell's genomic regulatory network as it is executing a programmatic change in its activities by combining biological pathway analysis with control theory engineering and mathematical segmentation analysis. The method also allows collection of data of the results of the information-processing in the cell by observing the decisions the cell makes when modulating cellular process activities.

In one embodiment, an in situ method for determining the types and levels of activity of cellular processes is disclosed, including determining the values for the activity of a regulatory element (e.g., a promoter) and the distribution of a localization reporter at time intervals over a period sufficient to ascertain whether cellular processes being monitored are stable under the culture conditions for promoter activity and localization reporter cellular distribution from at least one non-yeast eukaryotic cell transformed with at least one vector. Further, the at least one vector includes at least one cassette consisting of an inducible biological pathway specific promoter, where the promoter is operably linked to a first detectable marker, and at least one cassette consisting of a nucleic acid sequence encoding a first intracellular localization reporter. Moreover, cells transformed with the vector are subjected to external stimuli, and values are determined for the activity of the promoter and the distribution of the localization reporter repeatedly after exposure to a stimulus at time intervals over a period sufficient to follow the stepwise evolution of the cellular processes resulting from exposure to stimuli. Accordingly, a change in promoter activity and/or reporter localization is indicative of endogenous biological pathway modulation by the stimuli.

In one aspect, the determining of values includes ascertaining the values for the activity of a promoter and the distribution of a localization reporter in a panel of transformed non-yeast cells, where each cell contains a different vector comprising a separate and distinct pathway specific promoter. Further, the different cells in the panel exhibit separate and distinct responses to an applied stimuli, where differences in the cell processes arising from the differing constitutions of the cells in the panel that are initiated by each stimulus can be segregated and separately analyzed.

In a further aspect, the method includes analyzing time interval data using both data observed for a known biochemical pathway and model data for man-made network connectivity and process regulation to model connections between processes and regulatory conduits observed for the endogenous biological pathway. Such connectivity and process regulation include, but are not limited to, computer networks, communication systems/sub-systems, statistical process controls, and engineering process controls.

In another aspect, the method further includes applying state-space modeling to define control strategies to demonstrate the increase or decrease in the likelihood that a cellular process initiated by the stimulus would result in a perturbed cellular state or an unperturbed cellular state.

In one aspect, the method includes determining assay endpoints such as cell proliferation, cell senescence, and cell death.

In one aspect, the panel comprises from about 10 to 200 cells or more. In another aspect, the biological pathway is an endogenous or exogenous signaling pathway including, but not limited to, the PI3K/Akt/mTOR pathway.

In one aspect, determining the values is accomplished by image analysis, where the image analysis includes mathematical morphology segmentation, such as watershedding.

In a related aspect, the segmentation includes live staining the cells in a panel, locating separate signals from the live stain and fluorescence as a regionally thresholded binary image, combining the binary signals to produce a first merged image containing the thresholded binary images, placing marker lines at inflection points in valleys generated by the fluorescence signals to produce a second image, and combining the first merged image with the second image.

In one embodiment, a model generated by the mathematical morphology segmentation is disclosed.

### BRIEF DESCRIPTION OF THE DRAWINGS

FIG. 1 illustrates escape pathways that circumvent tumor dependence on the EGFR pathway mechanisms.

FIG. 2 shows a fluorescent image of nuclei in cells.

FIG. 3 shows an image of cells separated by watershed-based segmentation.

FIG. 4 shows an image of cells separated by applying watershed-based segmentation to thresholding results, where the segmented nuclei serve as markers for the presence of cells.

FIG. 5 graphically illustrates the application of watershed-based segmentation and thresholding results for various promoters in HEK and HT29 cells as a function of serum availability. Dashed lines show eGFP fluorescence levels in promoterless controls. Gray lines show eGFP fluorescence levels in serum starved cells. Dotted lines show eGFP fluorescence levels in cells continuously growing in 5% fetal bovine serum (FBS). Black lines show eGFP fluorescence levels in cells starved for 8 hours prior to addition of FBS to a final concentration of 20%.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a nucleic acid” includes one or more nucleic acids, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

A “vector” is a replicon, such as plasmid, phage or cosmid, to which another DNA segment may be attached so as to bring about the replication of the attached segment. A “vector” may further be defined as a replicable nucleic acid construct, e.g., a plasmid or viral nucleic acid.

An expression vector is a replicable construct in which a nucleic acid sequence encoding a polypeptide is operably linked to suitable control sequences capable of effecting expression of the polypeptide in a cell. The need for such control sequences will vary depending upon the cell selected and the transformation method chosen. Generally, control sequences include a transcriptional promoter and/or enhancer, suitable mRNA ribosomal binding sites and sequences which control the termination of transcription and translation. Methods which are well known to those skilled in the art can be used to construct expression vectors containing appropriate transcriptional and translational control signals. See, for example, techniques described in Sambrook et al., 1989, Molecular Cloning: A Laboratory Manual (2nd Ed.), Cold Spring Harbor Press, N.Y. A gene and its transcription control sequences are defined as being “operably linked” if the transcription control sequences effectively control transcription of the gene. Vectors of the invention include, but are not limited to, plasmid vectors and viral vectors. Preferred viral vectors of the invention are those derived from retroviruses, adenovirus, adeno-associated virus, SV40 virus, or herpes viruses. In general, expression vectors contain promoter sequences which facilitate the efficient transcription of the inserted DNA fragment and are used in connection with a specific host. The expression vector typically contains an origin of replication, promoter(s), terminator(s), as well as specific genes which are capable of providing phenotypic selection in transformed cells. Vectors suitable for use in the present invention include, but are not limited to the T7-based expression vector for expression in prokaryotes (Rosenberg, et al., Gene, 56:125, 1987), the ORFEX11 vector system (Ho et al., EMBO J, 6:133, 1987) or the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein I, cytomegalovirus immediate early, or polyhedrin promoters). The transformed hosts can be cultured according to means known in the art to achieve optimal cell growth conditions for the response to be examined.

A DNA “coding sequence” is a double-stranded DNA sequence which is transcribed and translated into a polypeptide in vivo when placed under the control of appropriate regulatory sequences. The boundaries of the coding sequence are typically determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters often, but not always, contain “TATA” boxes and “CAT” boxes. Prokaryotic promoters typically contain Shine-Dalgarno ribosome-binding sequences in addition to the −10 and −35 consensus sequences.

In one embodiment, the promoter is a biological pathway specific promoter. The term “biological pathway specific promoter” means a promoter which is modulated by one or more members of a set of interacting molecules and reactions that result in a select biological response or activity. For example, such pathways include, but are not limited to, metabolic pathways, signal transduction pathways, and gene regulatory pathways. In a related aspect, such promoters include, but are not limited to, PI3K pathway and cyclin D1 promoter; MEKK and c-jun promoter; cAMP/PKA pathway and ACE promoter, and the like.

Further, eukaryotic promoters include, but are not limited to, CMV immediate early, HSV thymidine kinase, early and late SV40, LTRs from retrovirus, human metallothionein IIA, HSP70, collagenase, α-2-macroglobulin, and mouse metallothionein-I. Selection of the appropriate vector and promoter is well within the level of ordinary skill in the art. The expression vector also contains a ribosome binding site for translation initiation and a transcription terminator. The vector may also include appropriate sequences for amplifying expression. Promoter regions can be selected from any desired gene using CAT (chloramphenicol transferase) vectors or other vectors with selectable markers.

In one aspect, the general method as disclosed allows data to be generated related to the activity of promoters in cells, and about the cellular location of macromolecules and second, of analytical techniques that use this information to infer the impact of cells' regulatory decisions on cellular processes and interpret this knowledge to determine optimal points in the process to manipulate to drive the cellular system to a desired state.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included near the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

A cell has been “transformed” by exogenous or heterologous DNA when such DNA has been introduced inside the cell. Transformation of a host cell with recombinant DNA may be carried out by conventional techniques as are well known to those skilled in the art. Such methods include, but are not limited to, calcium phosphate co-precipitates, conventional mechanical procedures such as microinjection, electroporation, insertion of a plasmid encased in liposomes, or virus vectors may be used. Another method is to use a eukaryotic viral vector, such as simian virus 40 (SV40) or bovine papilloma virus, to transiently infect or transform eukaryotic cells and express sequences of interest (see for example, Eukaryotic Viral Vectors, Cold Spring Harbor Laboratory, Gluzman ed., 1982).

The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or ancestor by mitosis. A “cell line” is a clone of a primary cell that is capable of stable growth in vitro for many generations.

Further, as such “host cells” are cells in which a vector can be propagated and its DNA expressed, the term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

The term “oligonucleotide”, as used herein, is defined as a molecule comprised of two or more deoxyribonucleotides, preferably more than three. Its exact size will depend upon many factors, which, in turn, depend upon the ultimate function and use of the oligonucleotide. The term “primer”, as used herein, refers to an oligonucleotide, whether occurring naturally (as in a purified restriction digest) or produced synthetically, and which is capable of initiating synthesis of a strand complementary to a nucleic acid when placed under appropriate conditions, i.e., in the presence of nucleotides and an inducing agent, such as a DNA polymerase, and at a suitable temperature and pH. The primer may initially be either single-stranded or double-stranded and must be sufficiently long to prime the synthesis of the desired extension product in the presence of the inducing agent. The exact length of the primer will depend upon many factors, including temperature, sequence and/or homology of primer and the method used. For example, in diagnostic applications, the oligonucleotide primer typically contains 15-25 or more nucleotides, depending upon the complexity of the target sequence, although it may contain fewer nucleotides.

The term “segmentation” means distinguishing between an object of interest and background in an image (e.g., distinguishing between foreground and background). In a related aspect, “thresholding” is a method of segmentation in which images are marked as “objects” if their value is greater than a set or threshold value (assuming an object to be brighter than the background) and as “background” if below this set value.

“Watershed transformation” is a tool for segmenting grayscale images, where the grayscale image is considered to be a topographical relief; i.e., the gray level of a pixel becomes the elevation of a point, the “basins” and “valleys” of the relief correspond to dark areas, where the “mountains” and “crest lines” correspond to light areas. The watershed line can be intuitively introduced as the set of points where a drop of water, falling there, may flow down towards several catchment basins of the relief (see, e.g., S. Beucher and Meyer, in Mathematical Morphology in Image Processing, E. R. Dougherty, Ed., New York: Marcell Dekker, 1993, vol. 12, pp. 433-481).

The term “in situ screening” means assaying cells without destroying/lysing cells to analyze subcellular components. In such an assay, the cells to be analyzed remain whole throughout the process.

The term “localization reporter” means a protein or protein fragment that contains a sequence which, when fused to a signal or other protein, sequesters the fused signal or other protein to specific organelles or subcellular structures. For example, such localization reporters include, but are not limited to, fusion proteins containing all or part of RhoB; subunit VIII of cytochrome c oxidase; SV40 T-antigen NLS; targeting sequence of calreticulin; targeting sequence from β1,4-galactosyltransferase; palmitoylation domain of neuromodulin; farnesylation sequence from hA-Ras; peroxisomal targeting signal; β-actin; AKT1; PLCG; histone H2B, and α-tubulin.

The term “time interval” involves the optimal magnitude of a choice variable in each period of a time within the observation period (e.g., discrete-time case) or at each time point in a given observation measure.

The term “network connectivity” means connecting and communicating between two or more nodes within a complex system, typically such connecting is over a series of points interconnected by one or more paths. In one aspect, the invention discloses the use of man-made networks and processes to model networks and processes in biological sub-systems. Such man-made models would include computer networks, communication systems/sub-systems, industrial polymerization processes involving integrating statistical process control (SPC) and engineering process control (EPC) combinations, and the like.

The term “process regulation” or “process control” involves the minimization of output variability in the face of dynamically related observations by making regular adjustments to one or more compensatory processing variables.

The terms “perturbed state” and “unperturbed state” relate to the equilibrium status of a given system. If a perturbation impinges on a system, and the system tends to return to its equilibrium, then the system is stable and in an unperturbed state. If a perturbation impinges on a system and the systems does not tend to return to its equilibrium, then the system is unstable and in a perturbed state. If a perturbation impinges on a system and the system does not move towards or away from equilibrium, then the system is in a neutral state.

The term “stepwise evolution” refers to a process of change marked by or proceeding in degree, grade, or rank in scale or involving a series of sequential changes in the stage of a system.

The term “state space modeling” refers to statistical methods for determining likelihood and probability, and include Markovian and non-Markovian models such as discrete-time Markov chains, continuous-time Markov chains, Markov reward models, semi-Markov models, Markov regenerative models, and non-homogenous Markov models.

Fluorescence labeling is a particularly useful tool for marking a protein, cell, or organism of interest. Traditionally, a protein of interest is purified, then covalently conjugated to a fluorophore derivative. For in vivo studies, the protein-dye complex is then inserted into cells of interest using micropipetting or a method of reversible permeabilization. The dye attachment and insertion steps, however, make the process laborious and difficult to control. An alternative method of labeling proteins of interest is to concatenate or fuse the gene expressing the protein of interest to a gene expressing a marker, then express the fusion product. Typical markers for this method of protein labeling include, but are not limited to, P-galactosidase, firefly luciferase and bacterial luciferase. These markers, however, require exogenous substrates or cofactors and are therefore of limited use for in vivo studies.

A marker that does not require an exogenous cofactor or substrate is the green fluorescent protein (GFP) of the jellyfish Aequorea Victoria, a protein with an excitation maximum at 395 nm, a second excitation peak at 475 nm and an emission maximum at 510 nm. Green fluorescent protein is a 238-amino acid protein, with amino acids 65-67 involved in the formation of the chromophore.

Uses of green fluorescent protein for the study of gene expression and protein localization are well known. The compact structure makes GFP very stable under diverse and/or harsh conditions such as protease treatment, making GFP an extremely useful reporter in general.

New versions of green fluorescent protein have been developed, such as a “humanized” GFP DNA, the protein product of which has increased synthesis in mammalian cells. One such humanized protein is “enhanced green fluorescent protein” (EGFP). Other mutations to green fluorescent protein have resulted in blue-, cyan- and yellow-green light emitting versions.

A broadly applicable tool devised for the study of cellular regulation is termed expression profiling (EP). Expression profiling allows the simultaneous determination of the relative amounts of RNA being expressed for many genes across a series of samples, providing a snapshot of the transcriptional activity of many of the genes in a cell at the time when the sample was taken.

EP utilizes destructive sampling. In order to make this kind of measurement, the cells to be studied must be broken into its macromolecular constituents, the RNA fraction purified from the other constituents, and then a labeled representation made of the RNA to serve as a quantifiable analyte. The requirement of destroying those cells to be analyzed leads to two of the key deficiencies of gathering data in this fashion, the difficulty of obtaining dynamic data and the averaging of the evolving data over a very large number of cells.

Successful analysis of the functioning of a complex system requires that one be able to follow the evolution of the system from one state to another in a very detailed way. If the method of following a system involves inactivating it, then one is faced with the necessity of having many, nearly identical systems going through the same set of state changes, and sacrificing systems at various times along the state evolution trajectory to be studied in order to build up a detailed picture of the various intermediate states the representative systems are passing though. This approach usually has practical limitations. The measurements quickly become very expensive, since you have to prepare many representative systems to undergo the test in a parallel, and you then have to carry out tests on a large number of systems. Choosing the time points for sampling is a very iterative process, since you do not have a precise temporal mapping of the events in state evolution at the outset, but have to learn this from the experiment.

In most of the experimentation in biology done to date, this problem is quite severe. While a small amount of data is taken in a time course, most data does not come from careful time-dependent experiments—or from timed experiments at all. In much of the transcription work carried out on human samples, the studies compare normal and pathological samples, each sample representing a single sampling taken from a single individual. Lacking time-course experiments, it is usually assumed that data come from the steady-state distribution of a system. Limited to steady-state data, the inference problem is an inverse problem of the following sort: given a sample from the steady-state distribution, what kind of inference can be obtained relative to the dynamics of the network. This inverse problem is strongly ill posed. Steady-state behavior constrains the dynamical behavior, but does not determine it. Building a dynamical model from steady-state data is a kind of overfitting. It is for this reason that the dynamical behavior of a network designed from steady-state data be viewed as an artifact, and that we must restrict ourselves to viewing the inferred network as providing a regulatory structure that must be interpreted so that the inferred network is restricted to that which is consistent with the observed steady-state behavior and with whatever biological assumptions are imposed upon the model, such as connectivity or attractor structure.

While it may be possible to capture the steady states of a biological network when modeling using steady-state data, many of the important operational characteristics of the network depend on its dynamical behavior. For instance, since attractor cycles depend on dynamical behavior, these cannot be determined by steady-state data and without dynamic characterization, it is typically necessary to postulate that the model only has non-cyclic steady-state behavior. Moreover, much important information is contained in the transient behavior of a network. For example, different attractor-compatible networks may have different transient behavior so that the behavior of the inferred network may or may not agree with known behavior, such as induction apoptosis by p53 or homeostatic behavior which requires a fast return to an attractor following perturbation. A more general problem relates to the steady-state probabilities. While good inference regarding the attractors may be obtained, only poor inference of the probability of the system ending in a given attractor after random initializations (or to random perturbations in more general networks) may be achieved. This is because if the basin of an attractor is small, it is less likely to catch a random initialization than if it is large. When sampling from the steady-state, attractors with small basins are less likely to appear, where those with large basins may appear numerous times.

The present invention describes intervening in the dynamics of a gene network by controlling one or more variables (or genes). In previous work, when inferring networks from steady-state data, it has been shown that control is exerted to the extent that the steady-state distribution is beneficially altered; however, the degree of alteration depends upon the particular model inferred from the steady-state data because, as was stated above, model inference lacking dynamical information has been selected to be consistent with the data, perhaps along with some prior biological assumptions and therefore may not provide an accurate gauge of the true distributions.

In addition, averaging, which is used frequently in methods that sample many cells, typically results in the loss of information in inferential analysis. At it simplest, averaging obscures the form of state changes that are taking place. The ability to discriminate between a digital, all-or-none change and an analog, graded change is lost. During a transition where cells will eventually achieve a change in transcription that can be represented as 1, it is impossible to know simply from an averaged measurement of 0.25 during the transition whether that represents all of the cells having changed by 25% of the full value, or 25% of the cells having changed by the full 100%. A method that gathered data on a cell-by-cell basis would produce this information, which could have an impact on how one would design an intervention targeting this transition.

A second kind of problem that arises from samples taken from multiple different individuals is that of correctly inferring the biological “meaning” associated with a change in the amount of transcript of a specific gene in a particular individual. It is well known that cells from different tissues react to the same signal in very different ways. Exposure of the whole body to strong gamma irradiation will have its most profound lethal effect on the very rapidly growing cells that produce blood cells and cells that line the gut, even though all cells have roughly equivalent dosage, and all cells will experience the immediate upregulation of many of the same set of stress-responsive genes. The difference in cell mortality is due to the ways that the initial regulatory changes will be subsequently translated into different responses, based on the type and amount of other gene products in the cell responding. In data sets that are gathered as single (or few) time point series, the differences in the interpretation of the informational elements by each of the different cell systems is not easily resolvable. Several possible errors could be made in attempting to associate the particular gene product with the process being studied. As biological systems exhibit the high degree of redundancy associated with stable complex systems, the participation of one particular gene product in a process might be dispensable for the overall success of the process. This became strikingly clear as it became possible to produce model organisms that completely lacked genes known to be important in carrying out specific developmental processes.

Frequently one would have to knock out several related genes to see effective blockage of the process in question. In addition to a gene being redundant, a gene can have multiple types of function that depends on what other genes are present and active in a cell. In this case, the gene's functional “meaning” is not fully inherent in the gene product but can only be fully determined by the context in which it is operating. Many proteins are activated or inhibited by post-translational modifications such as phosphorylation or by binding to another protein. In the case of redundancy, a protein could be absent and the process it is normally associated with could be ongoing. In the case of activation, a protein could be present across all the specimens and active in only the percentage of them where the protein was playing a role in the process of interest. Determining that a gene product is important in a process can therefore be difficult when working only from multiple sample comparisons.

The types of system studies described above have the elucidation of the network functions of normal cells in normal circumstances as their goal. In cases where one wishes to actively alter cells from their normal states, a typical goal for bio-production, or where one wishes to intervene in the activities of cells that are in an abnormal state, a typical problem for a physician dealing with pathological tissues, a very different perspective is called for. Observing normal cells in normal states does not reveal the large number of interlocking regulatory relationships that come into play when cells are subjected to unusual exposures, stresses or demands or when a cell's regulatory network is perturbed by mutation or genomic rearrangement, so that the ordinary, well-known relationships are not reliable modeling guides to what happens in abnormal circumstances. This has been a severe limitation in applied biology.

There has been a constant interest in the area of bio-production in being able to produce microbial or fungal strains altered to produce small molecules. Although many desirable molecules can be produced in these systems, the ability to drive the cells to produce large quantities of molecules such as the vitamin, oil and carotenoid derivatives of isoprene has been very limited, in spite of very explicit knowledge of the responsible enzymes, flux rates and the facile ability to put genes into and remove genes from the organisms' genomes. The problems appear at a higher level of the system, the interactions of the metabolic subsystem with other cellular subsystems that regulate the localization and storage of metabolic intermediates, and those that maintain the cell membrane.

Similarly, in attempting to devise drug interventions for cancer, even though one may know that a particular gene is stimulating chronic cell proliferation, treatments that eliminate that stimulus may fail to stop the tumor growth because other proliferative signals are operating in the system. In applications where the goal is to exert control on the cell regulatory systems to achieve a non-normal state or to alter a non-normal state, the focus of effort should first be adjusted upward in the regulatory hierarchy to examine the functioning of entire subsystems, to more clearly define the goals of the control.

In summary, in order to obtain the full power of existing engineering methods of analysis, it would be desirable to obtain data about cellular systems that would have the following properties:

1. The data is reflective of the dynamics of the system. It is gathered at sufficiently close intervals to allow the various state changes in the genomic regulatory system to be observed as the system evolved from one state to the next.

2. The data gathering method is non-destructive, and allows one to follow the course of a biological program in the same sets of cells.

3. The data gathering method should be practical with modest amounts of cells so that specific tissues from organisms can be obtained and tested.

4. The data should be collected on a cell-by-cell basis so that the coherence and extent of change across the population can be determined.

5. Corollary data identifies the functional status of the various processes being studied should also be gathered, so that the actual effects of the stepwise changes on the process can be studied.

One approach to obtain data with these characteristics is to carry out experiments that allow observation of the functioning of a variety of cell subsystems before control is attempted, to determine how they are being controlled in their starting state, and the applying the control intervention and watching the evolution the functional status of the subsystems to see whether the control affected the target it was designed for, also whether an effective change of the target's function produced the expected changes in the other components of the subsystem and in the subsystems that interact with the targeted subsystem. If the control fails, it would be possible to see that it failed because there was an alternative source of the function that restored the targeted subsystem to functionality or because there was an alternative source of function that could replace the input of the targeted subsystem on the other subsystems it normal interacts with. The types of the interactions that arise in these contexts are unlikely to have been encountered in the steps that build up a deep understanding of the regulatory relationships that are the basis of normal function.

Thus, features of the disclosed method are:

1. Each cell line is independently tested for drug response and full data about its status prior to drug exposure and after drug exposure is taken.

2. Many pathway operations and interactions will be examined simultaneously

3. The data points are taken at short intervals (˜15 minutes) over the entire pre and post treatment time span, allowing all of the intermediate steps in the response to be captured.

4. Data is taken cell by cell, not as a single average of all cells tested.

5. Some indicators will allow direct visual assessment of proliferation and apoptosis.

6. A variety of cell lines or tumors are examined to sample the many possible tumor contexts in which the drug might be used.

7. The particular molecular response events that occur when the drug is successful and when it fails in each cell line will be available for cross comparison.

8. The chains of interaction necessary for a successful response can be identified.

9. Antagonistic processes can be identified.

In one embodiment, an in vivo method for determining the types and levels of activity of cellular processes is disclosed including determining the values for the activity of a promoter and the distribution of a localization reporter repeatedly at time intervals over a period sufficient to ascertain whether cellular processes being monitored are stable under the culture conditions for promoter activity and localization reporter cellular distribution from at least one non-yeast eukaryotic cell transformed with at least one vector, where the at least one vector includes at least one cassette consisting of an inducible biological pathway specific promoter, where the promoter is operably linked to a first detectable marker and at least one cassette consisting of a nucleic acid sequence encoding a first intracellular localization reporter; subjecting the transformed cell to external stimuli; and determining the values for the activity of the promoter and the distribution of the localization reporter repeatedly after exposure to a stimulus at time intervals over a period sufficient to follow the stepwise evolution of the cellular processes resulting from exposure to stimuli, where a change in promoter activity and/or reporter localization is indicative of endogenous biological pathway modulation by the stimuli.

In a related aspect, determining includes ascertaining the values for the activity of a promoter and the distribution of a localization reporter in a panel of transformed non-yeast cells, wherein each cell contains a different vector comprising a separate and distinct pathway specific promoter, whereby the different cells are able to exhibit separate and distinct responses to an applied stimuli, and wherein differences in cell processes initiated by each stimulus can be segregated and separately analyzed.

In one aspect, the method includes analyzing time interval data using both data observed for a known biochemical pathway and model data for man-made network connectivity and process regulation to model connections between processes and regulatory conduits observed for the endogenous biological pathway.

In another aspect, the method further includes applying state-space modeling to define control strategies to demonstrate the increase or decrease in the likelihood that a cellular process initiated by the stimulus would result in a perturbed cellular state or an unperturbed cellular state.

These methods may involve, but are not limited to, mathematical control or control engineering theory. To control an object means to influence its behavior so as to achieve a desired goal. Generally, there have been two main lines of work in control theory. One of these is based on the idea that a good model of the object to be controlled is available and that one wants to some how optimize its behavior. The other main line of work is based on the constraints imposed by uncertainty about the model or about the environment in which the object operates. The central tool for this type of modeling is the use of feedback in order to correct for deviations from the desired behavior (e.g., observations from perturbed and unperturbed states).

For the present invention, certain principles from control theory can be applied. For example, first-order approximations are sufficient to characterize local behavior. Based on the linearization principle, models based on linearizations work locally for the original system. The term “local” refers to the fact that satisfactory behavior can be expected for those initial (e.g., unperturbed) states that are close to the point about which the linearization was made. A more in-depth analysis of control theory can be found in F. L. Lewis, Applied Optimal Control and Estimation, Prentice-Hall, New York, N.Y., 1992 and E. D. Sontag, Mathematical Control Theory: Deterministic Finite Dimensional Systems, Second Edition, Springer-Verlag, New York, N.Y., 1998.

In one aspect of the invention, a determining step involves determining values for the activity of a promoter and the distributions of a localization reporter repeatedly after exposure to a stimulus at time intervals over a period of time sufficient to follow the stepwise evolution of the cellular processes resulting from exposure to stimuli, wherein a change in promoter activity and/or reporter localization is indicative of endogenous biological pathway modulation by the stimuli. For example, increase or decrease in the activation of a component of a biological pathway can be analyzed in a linearized model.

Recent advances in molecular oncology have generated optimism that the development of cancer drugs and selection of treatments can be transformed from a tissue pathology and population guided approach to one that is target driven. There is compelling evidence that activating mutations in signaling pathways can result in tumor cell “addiction” to a pathway resulting in the expectation that drugs developed to inhibit these pathway will lead to tumor death. It has become clear, however that tumor cell responses to drugs designed to inhibit a particular pathway are conditioned by a large number of cellular activities that are independent of that one step. This has led to the understanding that the much broader cellular context must be evaluated to determine the conditions under which the drug will produce the desired response. The most common approaches to assessing cellular context currently are a sampling of the tumor prior to treatment. The tumor context is then assessed via complex and expensive experiments including Western Blot, mRNA microarrays, etc. The resulting data is a one-time snapshot of mRNA abundance levels and protein abundance/modification levels that is an average over many cells.

In accordance with this invention, images of cells are manipulated and analyzed in certain ways to extract relevant biological pathway-related features. Using those features, the apparatus and processes of this invention, can automatically draw certain conclusions about the biology of a cell.

The invention provides methods and apparatus that for the analysis of images of cells and extraction biologically-significant pathway-related features from the cell images. The extracted features may be correlated with particular conditions induced by biologically-active agents (e.g., drugs, peptides, proteins, nucleic acids, infectious agents, hormones, small organic molecules, inorganic molecules, metals, organic-metal conjugates, antigens, antibodies, chemokines, cytokines, carbohydrates, lipids, vitamins, and the like) with which cells have been treated or physical agents (e.g., heat, light pressure, magnetic fields, X-radiation, or non-thermal microwave radiation), thereby enabling the automated analysis of cells based on pathway utilization parameters. In particular, the invention provides methods for segmentation of cells in an image using data from a plurality of separate images.

One application of the invention involves the use of a reference cell pathway (preferably one where the indicative features of the cellular image have been previously identified and segmented and therefore one whose identification and segmentation parameters are well understood and may be repeated) in combination with image data to perform segmentation on a second cell to obtain data about the pathway or subsystem of the second cell. This application of the invention is particularly effective when reference cell features (e.g., cytoplasm, nucleus, mitochondria, endoplasmic reticulum, cytoskeleton, or other visualizable feature) have been previously segmented. The invention further provides techniques for extraction of biologically-relevant pathway-related cell features from segmented cell images.

In accordance with the present invention, images may be obtained of cells that have been treated with a chemical agent to render visible (or otherwise detectable in a region of the electromagnetic spectrum) components of cell subsystems and/or localization or specific sequestration (e.g., translocation) of markers into subcellular compartments. A common example of such agents are colored dyes specific for a particular cellular component that is indicative of cell shape. Other such agents may include fluorescent or phosphorescent compounds that bind directly or indirectly (e.g., via antibodies or other intermediate binding agents) to a cell component. In accordance with the present invention, a plurality of cell components may be treated with different agents and imaged separately, so long as the agents do not distort the cellular response of interest.

Generally the images used as the starting point for the methods of this invention are obtained from cells that have been specially treated and/or imaged under conditions that contrast markers from other cellular components and the background of the image. In one embodiment, the cells are treated with a live cell stain that produces a distinct visible marking of each cell in an image. In one aspect, the chosen imaging agent binds indiscriminately to or within the cell. The agent should provide a strong contrast to other features in a given image. To this end, the agent should be luminescent, fluorescent, and the like. Various stains and fluorescent compounds may serve this purpose.

A variety of imaging agents are available depending on the particular marker, and agents appropriate for labeling cytoskeletal, cytoplasmic, plasma membrane, nuclear, and other discrete cell components are well known in the histology and cell biology art.

Various techniques for preparing and imaging appropriately treated cells are well known in the art (see, e.g., U.S. Pat. No. 6,734,576).

In each case, the image obtained will represent the imaged marker as a corresponding “image parameter.” The image parameter will be an intensity value of light or radiation shown in the image. Often, the intensity value will be provided on a per pixel basis. In addition, the intensity value may be provided at a particular wavelength or narrow range of wavelengths that correspond to the emission frequency of an imaging agent that specifically associates with the imaged marker.

Sometimes corrections must be made to the measured intensity. This is because the absolute magnitude of intensity can vary from image to image due to changes in the staining and/or image acquisition procedure and/or apparatus. Specific optical aberrations can be introduced by various image collection components such as lenses, filters, beam splitters, polarizers, etc. Other sources of variability may be introduced by an excitation light source, a broad band light source for optical microscopy, a detector's detection characteristics, etc. Even different areas of the same image may have different characteristics. For example, some optical elements do not provide a “flat field.” As a result, pixels near the center of the image have their intensities exaggerated in comparison to pixels at the edges of the image. A correction algorithm may be applied to compensate for this effect. Such algorithms can be easily developed for particular optical systems and parameter sets employed using those imaging systems. One simply needs to know the response of the systems under a given set of acquisition parameters.

The concepts underlying thresholding are well known. An appropriate threshold may be calculated by various techniques. In a specific embodiment, the threshold value is chosen as the mode (highest value) of a contrast histogram. In this technique, a contrast is computed for every pixel in the image. The contrast may be the intensity difference between a pixel and its neighbors. Next, for each intensity value (0-255 in an eight byte image), the average contrast is computed. The contrast histogram provides average contrast as a function of intensity. The threshold is chosen as the intensity value having the largest contrast. See “The Image Processing Handbook,” Third Edition, John C. Russ 1999 CRC Press LLC IEEE Press, and “A Survey of Thresholding Techniques,” P. K. Sahoo, S. Soltani and A. K. C. Wong, Computer Vision, Graphics, and Image Processing 41, 233-260 (1988). In one embodiment, edge detection may involve convolving images with the Laplacian of a Guassian filter. The zero-crossings are detected as edge points. The edge points are linked to form closed contours, thereby segmenting the relevant image objects. See The Image Processing Handbook, referenced above. Further details regarding the segmentation of nuclei in accordance with the present invention and associated apparatus and techniques are described in co-pending patent application Ser. Nos. 09/729,754 and 09/792,012 (Publication No. 20020141631).

Digital images can be processed in conjunction with each other using a watershed technique in order to achieve segmentation of the cells in the original image. The concepts underlying watershed algorithms are well known. The topology of cells can be represented as peaks and valleys of various magnitudes. The high peaks represent the points at which valleys ultimately meet; by way of analogy, the point at which bodies of water rising from springs (referred to as “seeds” in watershed terminology) at the base of a valley would meet, and thus represents the ultimate boundary of a valley, where the top of a high peak is referred to as a “watershed.”

Appropriate watershed algorithms suitable for use in accordance with the present invention are described in detail in L. Vincent and P. Soille, Watersheds in digital spaces: an efficient algorithm based on immersion simulations, IEEE Transactions on Patter Analysis and Machine Intelligence, 13:583-589, 1991.

At some point, an image analysis process must obtain image parameters relevant to a biological condition of interest. Typically, the parameters of interest relate to the size, shape, contour, and/or intensity of the cell images. Examples of some specific parameters for analysis include the following:

| | |
|---|---|
| Total Intensity | (sum of pixel intensities in an object) |
| Average Intensity | (average intensities in an object) |
| Area | (number of pixels in an object) |
| Axes Ratio | (ratio of lengths of axes of a fitted ellipse) |
| Eccentricity | (distance from the center of an ellipse to its focus) |
| Solidity | (measure of pixels inside versus pixels outside an |
| | object surrounded by a simple shape) |
| Extent | (the area of the object divided by area of the smallest |
| | box to contain the object) |
| X_coord | (the X coordinate of an object's centroid) |
| Y_coord | (the Y coordinate of object's centroid) |
| Form Factor | (characteristic of the shape of the outline of an object) |
| Diameter | (the equivalent diameter of an object, that is the |
| | diameter of the circle with the same area as the object) |
| Moment | (characteristic of the shape of an outline of an object, |
| | also taking into account the distribution of pixels |
| | inside the object) |

Image analysis routines for extracting these various parameters and others can be designed using well known principles. See The Image Processing Handbook, referenced above. In addition, various commercially available tools provide suitable extraction routines. Examples of some of these products include the MetaMorph Imaging System, provided by Universal Imaging Corporation, a company with headquarters in West Chester, Pa. and NIH Image, provided by Scion Corporation, a company with headquarters in Frederick, Md.

Other well known techniques employ skeletonization, and techniques for the computation of end points and nodes, from an object's skeleton are well known. See, for example, J. C. Russ, The Image Processing Handbook, CRC press, 1998.

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

The following examples are intended to illustrate but not limit the invention.

### EXAMPLES

### Example 1

### Assays of Living Cancer Cells Prior and after Exposure to a Cancer Drug

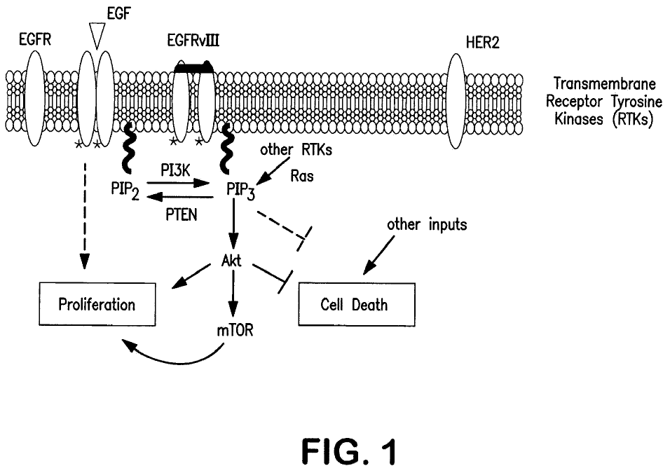

In trials treating patients with EGFR kinase inhibitors, it has been seen that tumors profiled by typical pre-treatment methods showed molecular signs consistent with dependence on EGFR kinase pathway and some signs of sensitivity to EGFR kinase inhibitors. Even with these findings, most of the patients' tumors were resistant to the treatment, despite initial responsiveness. It is now felt that the reason for this phenomenon is that there are one or more escape pathways that circumvent the tumors' addiction to the EGFR pathway. This is illustrated in FIG. 1.

Observations showing that a deficiency or mutation of PTEN is common in resistant patients led to hypothesis that this condition is circumventing the EGFR dependency, and causing resistance to EGFR kinase pathway inhibitors. PTEN is an antagonist to PI3K's effect of phyosphorylating PIP2 to PIP3. High concentrations of PIP3 drive the Akt/mTOR pathways which are themselves capable of promoting tumor cell proliferation and cell survival and this PIP3 accumulation is facilitated when PTEN is deficient.

Using our characterization of tumor cell responses, it is possible to see the failure of the EGFR kinase inhibitor to stop cell proliferation and the active operation of the PTEN deficiency induced Akt/mTOR pathway in PTEN deficient cells and the success of the EGFR kinase inhibitor in stopping cell proliferation and the inactive status of the Akt/mTOR pathway in PTEN proficient cells, as well as other pathways involved in inducing EGFR inhibition resistance in cell lines representative of these contexts.

Measurements of promoter activity in vivo have been developed that exploit variants of a green fluorescent protein (GFP) first isolated from jellyfish. A recombinant plasmid is generated that contains GFP under transcriptional control of a promoter cloned from an organism. This construct is delivered back to cells from the organism (or into an intact organism) and provides a quantifiable marker of the promoter's activity. In addition to quantitative information, the fluorescent protein yields information about the intracellular location of proteins, and how this changes as they are modified.

A fluorescent protein is fused to the amino or carboxy terminus of another protein to allow the use of fluorescent microscopy to examine the fusion protein's distribution in the cell. By making ubiquitously expressed fluorescent protein fusions or even protein domains that are localized to macromolecules whose cellular localization and distribution are important markers for particular cellular processes such as mitosis, or localized phosphorylation of lipid, dynamic reports are obtained that show how the induction or reduction of cellular processes affect other cellular processes.

Cells known to respond in varying ways to a particular stimulus are partitioned into culture wells in a multiwell plate suitable for backside microscopy at a low density (20% confluence). The cells in each culture well are transfected with a particular type of reporter (promoter and/or localization), using viral particle packaging/delivery, chemical, or electroporation methods.

With methods having lesser efficiencies, the cell populations would have to be selected for stable transformation on the basis of a selectable antibiotic resistance gene carried on the same vector as the promoter and reporter. The transformed cells are then to be examined to determine the basal levels of activity of the promoter reporters and the baseline distribution of the cellular localization reporters. After obtaining a series of baseline measurements, the cells are subjected to a stimulus of interest, and a further series of measurements are taken at short enough intervals to allow the details of the response to be captured (˜15 minutes).

The image data is then analyzed to obtain quantitative information from the promoter reporters and process engagement information from the localization probes. Two images for two different fluorescent channels are acquired from one area within each well element: e.g., a red channel is used for Vybrant® DyeCycle™ Orange stain which is used as a live stain for nuclei (a histone fusion to a red fluorescent protein can also be used) and the green for the GFP reporter signal. The cells in the fluorescent image are segmented to identify all the individual cell areas and the images are analyzed to extract multiple parameters from each individual cell area (including the minimum, maximum, mean, median and total fluorescence intensity). Interpretation of the localization reporters is carried out with morphology filters, or may be directly interpreted by the investigator.

The images that will be assessed for promoter activity are processed via a watershed-based multi-step process to generate binary image masks for measuring fluorescence intensities in each channel for segmented cells. The steps in the image analysis are: (1) the Vybrant® DyeCycle™ Orange stain image and the green image are regionally threshold to generate binary images. (2) These two binary images are combined to produce a merged image of the union of the two thresholds. (3) The green image is analyzed to determine the graduations in the green signal intensity gradients and watershed lines are placed at the inflection points in the valleys of this green signal using the Vybrant® DyeCycle™ Orange stain derived binary image as a marker. (4) The merged binary image in step 2 is now combined with the green image subjected to water-shedding to locate and define the precise area of every cell in the image. (5) Partial cells and noise are subtracted to produce a fully segmented image suitable for extraction of multi-parametric data. Statistical analysis of the multi-parametric data including generation of global statistics of cell population in each image is performed and local background is calculated and subtracted from the global statistics.

The promoter intensity data is analyzed to determine whether the observed behavior is consistent with the networks that have been proposed to control the processes examined. To the extent that the behavior appears concordant, a model will be built that reflects this prior knowledge. Where the observations are at variance with the known network, adjustments will be made to produce novel network segments that are consistent with the observation.

A number of methods to design optimal control policy based on the network structure and rules in biological systems have been designed. Those that will be applied initially will be variations of these methods that are customized for time course data.

### Example 2

### Measuring Promoter Responses

In order to observe cellular processes invoked by a particular stimulus and differences in the processes that are invoked in differing cell, experiments were carried out so that one or more promoters' responses may be tracked by the fluorescent protein expression.

For each promoter of interest, an artificial construct was produced which places the coding sequence of a fluorescent protein under the control of a specific promoter. For this example, a lentiviral system (Invitrogen Gateway pLenti6/R4R2/V5-DEST) was used, which allows for rapid, modular, combinatorial assembly of promoters and reporters. Three sequences from the promoter region of the genes for EGR1 (i.e., early growth response 1; SEQ ID NO: 1), MYC (v-myc myelocytomatosis viral oncogene homolog; SEQ ID NO:2), and JUN (jun oncogene; SEQ ID NO:3) were recovered from normal human DNA by PCR amplification and directionally cloned into pENTR™5′-TOPO (Invitrogen) plasmids. A fluorescent reporter protein, eGFP (enhanced green fluorescent protein; SEQ ID NO:4) was recovered from pCMV GIN-ZEO (Open Biosystems) and cloned into pENTR11 (Invitrogen) plasmid. Reporter constructs were assembled by recombination of the three plasmids. One plasmid (pENTR™5′-TOPO plus promoter sequence) contains the promoter sequence. A second plasmid (pENTR11 plus reporter coding sequence) contains the fluorescent protein coding sequence. The third plasmid (pLenti6/R4R2V5-DEST) contains the lentiviral packaging and chromosomal integration signals for delivery of the promoter reporter to chromosomes in the target cells, sequences that allow the recombination with the two other plasmids to assemble the promoter and a coding sequence in a configuration that allows the promoter to drive transcription of the coding sequence, and a gene conferring resistance to the drug blastocidin to allow selection of cells to which the reporter was delivered.

The resulting recombined plasmid product can be used to exploit the efficiency of packaging the reporter constructs as lentiviral particles to deliver the reporter constructs to the cells to be assayed for promoter response. The recombined plasmid and helper plasmids that supply other proteins required for packaging are transfected into a 293FT cell line to produce viral particles that can efficiently deliver constructs into most cells (Invitrogen, ViraPower™ II Lentiviral Gateway™ Expression kit). Once established, lines with these reporters are monitored in real time for their response to various drugs and other stimuli.

For the present example, the human embryonic kidney cell line, HEK (near normal), and colon cancer cell line, HT29, were used to assay for the responses of the EGR1, MYC, and JUN reporters to a period of serum deprivation followed by a period of renewed exposure to serum. The serum response of cells is typically characterized by removing one of the normal constituents of the media used to culture cells in vitro; e.g., fetal bovine serum (FBS). FBS is very rich in growth factors, and supports growth of cells in culture. Generally, cells can live for a few days without FBS, however, they cease growing and will eventually die in the absence of the supplement. Normally, cells that have been deprived of serum for 8 to 16 hours, followed by re-exposure to serum, have a fairly characteristic response that includes rapid induction of transcription of a number of “serum-responsive” genes (Iyer et al., Science (1999) 283(5398):83-87). A typical member of this family of responsive genes is EGR1. The promoter for this gene was placed so that it would drive the production of fluorescent protein, eGFP, in the lentiviral cloning system as described. Further, this same mechanism would drive the promoters for the genes JUN and MYC, although these genes are more variably responsive to serum. EGR1, JUN, MYC are all themselves transcription factors, and therefore capable of producing further widespread, cascading transcriptional changes.

The placement of promoter reporter and control cells (without a promoter reporter) on a 96-well culture plate is shown in the diagram below.

| | | | | | | | | | | | | | Serum | Serum Re- |
|---|---|---|---|---|---|---|---|---|---|---|---|---|---|---|
| | HEK | | | | | | HT29 (Colon Cancer) | | | | | | Starvation | Feeding |
| | 1 | 2 | 3 | 4 | 5 | 6 | 7 | 8 | 9 | 10 | 11 | 12 | Status | Status |
| A | JUN | JUN | JUN | JUN | Con | Con | JUN | JUN | JUN | JUN | Con | Con | Starve 8 | 20% FBS |
| B | MYC | MYC | MYC | MYC | Con | Con | MYC | MYC | MYC | MYC | Con | Con | Hours | |
| C | EGR1 | EGR1 | EGR1 | EGR1 | Con | Con | EGR1 | EGR1 | EGR1 | EGR1 | Con | Con | | |
| D | JUN | JUN | JUN | JUN | Con | Con | JUN | JUN | JUN | JUN | Con | Con | | No FBS |
| E | MYC | MYC | MYC | MYC | Con | Con | MYC | MYC | MYC | MYC | Con | Con | | |
| F | EGR1 | EGR1 | EGR1 | EGR1 | Con | Con | EGR1 | EGR1 | EGR1 | EGR1 | Con | Con | | |
| G | JUN | MYC | EGR1 | | Con | Con | JUN | MYC | EGR1 | | Con | Con | Don't | N/A |
| H | JUN | MYC | EGR1 | | Con | Con | JUN | MYC | EGR1 | | Con | Con | Starve | |

The design provides 4 replicates of each reporter and 6 of each non reporter control for the starvation and replenishment series and 2 replicates of each reporter and 4 of each non-reporter for the continuous FBS exposure series.

Three kinds of pretreatment were performed before the cells were imaged. Cells were plated at about 6000 cells per well and grown for 30 hours in media with 10% FBS. Cells in plate rows A-C and D-F were all subjected to eight hours of serum deprivation. Cells in plate rows G-H were not starved. Just prior to imaging, the media for cells in plate rows A-C was changed to media plus 20% FBS. The classes of treatment were continuous FBS (G-H), FBS deprivation (D-F) and FBS deprivation then FBS replenishment (A-C). Imaging of the wells at twenty-minute intervals using an InCell 3000 automated laser excitation, confocal imaging instrument (General Electric) commenced immediately after adding FBS. Nuclear fluorescence was monitored based on the production of the green fluorescent protein, eGFP.

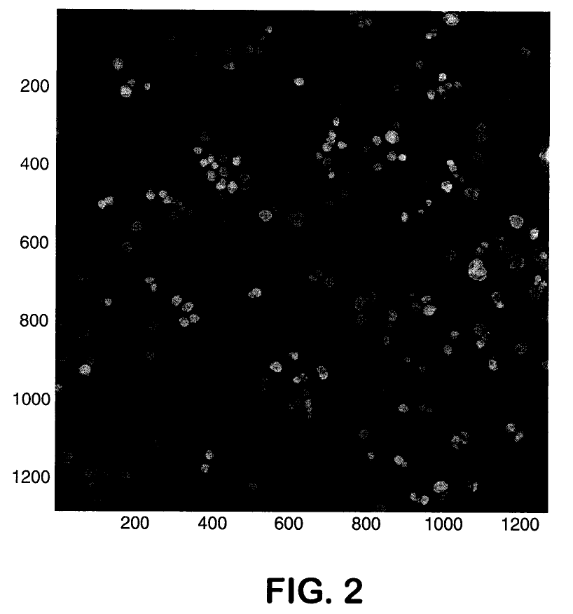

To obtain the fluorescent intensity in each cell, the images were processed to extract the necessary information. A typical fluorescent image is shown in FIG. 2. The intensity readings from the channel recording the nuclear fluorescence emission is shown as dark gray. The intensity of the channel recording the eGFP fluorescence emission is shown as light gray. Traditional image processing methods based on signal intensity levels and the morphology of the cellular regions of interest are applied to obtain qualification of the speed and extent of changes in eGFP production driven by the promoter being assayed.

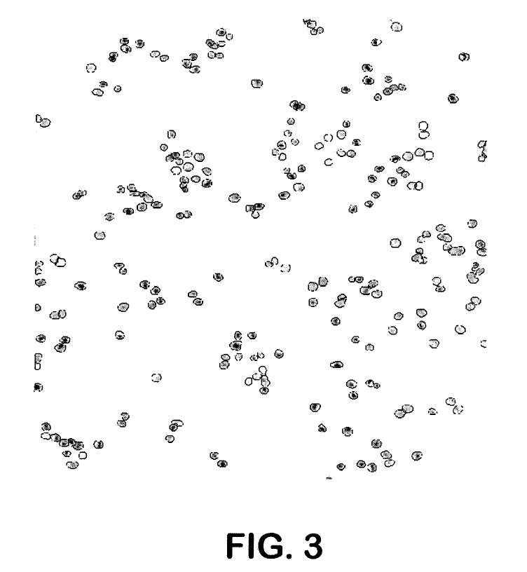

First, the nuclei channel is processed to locate all nuclei present in the image. A morphological filter method, the open top-hat transform, is applied to the image. A round kernel with size slightly larger than the normal nuclei size is chosen to filter out objects that are unlikely to be nuclei. The top-hat segmented results are then polished by morphological opening with a small kernel, followed by area opening to remove small debris. To further separate individual cells, watershed-based segmentation is applied to the polished top-hat segmentation results, where local maximum of the smoothed nuclei channel images are used as markers. The segmented results are shown in FIG. 3, where the intensity values are negated for better viewing.

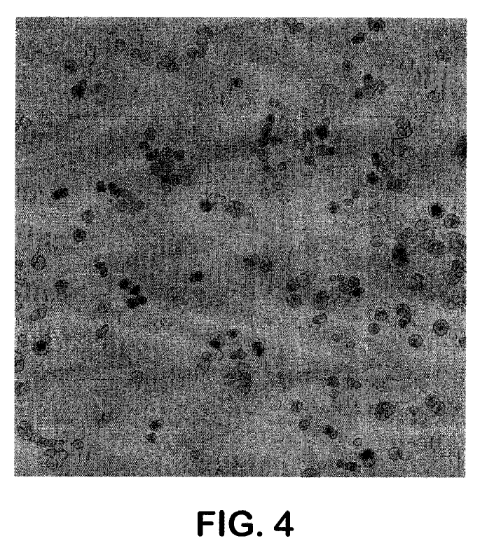

Next, the eGFP channel is processed. First, a global threshold is applied to the image to detect all signals above threshold. The thresholding results are then polished by two rounds of morphological opening and closing to remove noise, followed by area opening to remove small debris. To further separate individual cells, watershed-based segmentation is applied to the thresholding results, allowing the segmented nuclei serve as the markers of the presence of a cell. The segmented results are shown in FIG. 4, where the intensity values are inverted (dark is more intense, light is less) for better viewing.

The segmentation results naturally link each nucleus to its associated area of eGFP fluorescence. For average eGFP intensity, the total eGFP intensity should first have the background intensity subtracted, before it is normalized by nuclei count, or any equivalent measurement.

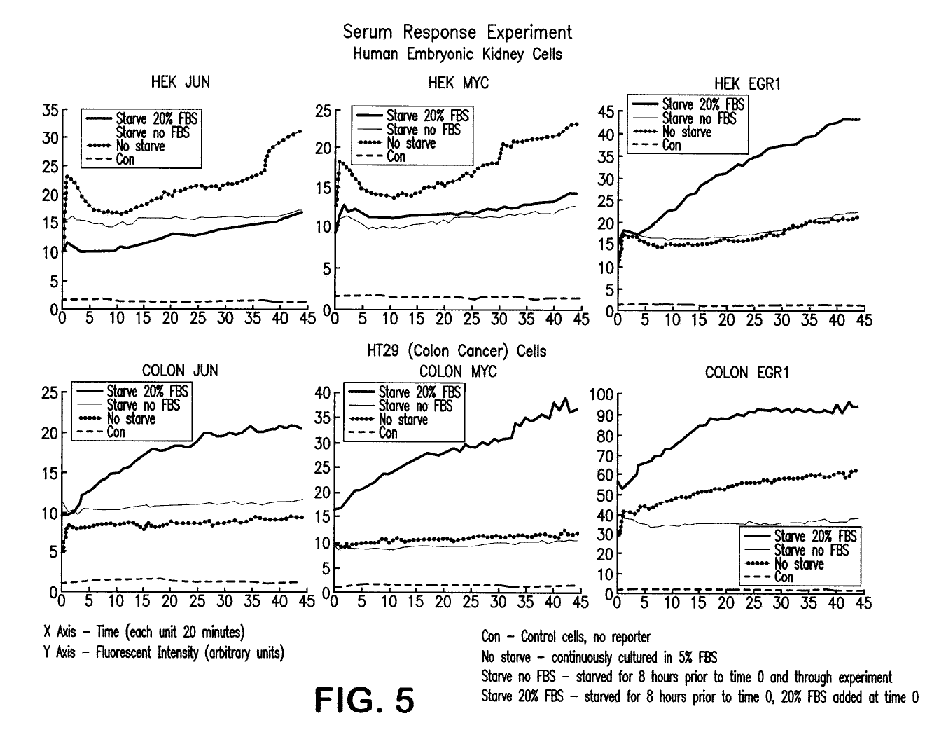

After applying this analysis to determine average eGFP intensities for all of the images taken during the course of the experiment, plots of these values over the course of the experiment for all of the promoter reporter containing and promoter reporterless versions of the HEK and HT29 cells were prepared. These are shown in FIG. 5. These graphs show that for all cases, the relevant promoter reporterless controls show eGFP fluorescence levels that are uniform and near zero (dashed lines). Continuously serum deprived (starved) cells show uniform, unchanging behavior across all cell types (gray lines). (Note that the first point taken by the machine has some variability due to a mechanical problem specific to the first image of a series). Continuously growing cells have no or slowly rising levels of production of eGFP (dotted lines). Both HT29 and HEK cells deprived of serum and then re-exposed to serum (black lines) show a similar response for the EGR1 promoter, a rapid rise and leveling off of eGFP production, as expected. HT29 and HEK cells bearing the MYC and JUN promoters showed considerable differences in their response to serum starvation. The HT29 cells had a rapid, more substantial increase in eGFP production, while the HEK cells had a very modest and gradual rise. This indicates a significantly different pattern of cellular response in activation of the proliferative process of the HT29 cells relative to HEK cells, demonstrating the ability of the present technique to differentiate the ways that cellular processes respond to particular stimuli.

Although the invention has been described with reference to the above examples, it will be understood that modifications and variations are encompassed within the spirit and scope of the invention. Accordingly, the invention is limited only by the following claims.

We claim herein:

1. An in situ method for determining the types and levels of activity of cellular processes comprising:
a) determining the values for the activity of a promoter and the distribution of a localization reporter repeatedly at time intervals over a period sufficient to ascertain whether cellular processes being monitored are stable under the culture conditions for promoter activity and localization reporter cellular distribution from at least one non-yeast eukaryotic cell transformed with at least one vector, wherein the at least one vector comprises:
i) at least one cassette consisting of an inducible biological pathway specific promoter, wherein the promoter is operably linked to a first detectable marker, and
ii) at least one cassette consisting of a nucleic acid sequence encoding a first intracellular localization reporter;
b) subjecting the transformed cell to external stimuli; and
c) determining the values for the activity of the promoter and the distribution of the localization reporter repeatedly after exposure to a stimulus at time intervals over a period sufficient to follow the stepwise evolution of the cellular processes resulting from exposure to stimuli, wherein a change in promoter activity and/or reporter localization is indicative of endogenous biological pathway modulation by the stimuli.

2. The method of claim 1, further comprising analyzing the time interval data using both data observed for a known biochemical pathway and model data for man-made network connectivity and process regulation to model connections between processes and regulatory conduits observed for the endogenous biological pathway.

3. The method of claim 2, wherein determining step (a) comprises determining the values for the activity of a promoter and the distribution of a localization reporter in a panel of transformed non-yeast cells, wherein each cell contains a different vector comprising a separate and distinct pathway specific promoter, whereby the different cells exhibit separate and distinct responses to an applied stimuli, and wherein differences in cell processes initiated by each stimulus can be segregated and separately analyzed.

4. The method of claim 3, further comprising applying state-space modeling to define control strategies to demonstrate the increase or decrease in the likelihood that a cellular process initiated by the stimulus would result in a perturbed cellular state or an unperturbed cellular state.

5. The method of claim 1, further comprising determining assay endpoints selected from the group consisting of cell proliferation, cell senescence, and cell death.

6. The method of claim 1, wherein the promoter is endogenous or exogenous to the cell.

7. The method of claim 1, wherein the vector is a plasmid vector or a viral vector.

8. The method of claim 1, wherein the detectable marker is a fluorescent protein.

9. The method of claim 8, wherein the fluorescent protein is luciferase or green fluorescent protein (GFP).

10. The method of claim 1, wherein the panel comprises from about 10 to 200 cells.

11. The method of claim 1, wherein the biological pathway is an endogenous or exogenous signaling pathway.

12. The method of claim 11, wherein the biological pathway is the PI3K/Akt/mTOR pathway.

13. The method of claim 1, wherein the cell is a non-neoplastic or a neoplastic cell.

14. The method of claim 1, wherein the localization reporter is translocated to the inner cell membrane, to the nucleus, to the golgi apparatus, to the mitochondria, to the endoplasmic reticulum, sequestered in the cytoplasm, or a combination thereof.

15. The method of claim 1, wherein determining the values is accomplished by image analysis.

16. The method of claim 15, wherein image analysis comprises mathematical morphology segmentation.

17. The method of claim 16, wherein the segmentation comprises:
a) live staining the cells in the panel;
b) locating separate signals from the live stain and fluorescence as a regionally thresholded binary image;
c) combining the binary signals to produce a first merged image comprising the thresholded binary images;
d) placing marker lines at inflection points in valleys generated by the fluorescence signals to produce a second image; and
e) combining the first merged image with the second image.

18. The method of claim 17, wherein the mathematical morphology segmentation is accomplished by watershedding.

19. The method of claim 17, further comprising comparing promoter activity data obtained from step (e) to model data observed for a known biochemical pathway and adjusting any variances between the model data from the known pathway and data obtained from the assay data.

20. A model generated by the method of claim 19.

21. The method of claim 1, wherein the external stimuli is exposure to a chemical or physical agent.

22. The method of claim 21, wherein the chemical agent is a peptide, a protein, a nucleic acid, a bacteria, a virus, a hormone, a small organic molecule, an inorganic molecule, a metal, an organic metal conjugate, an antigen, an antibody, a chemokine, a cytokine, a carbohydrate, a lipid, or a vitamin.

23. The method of claim 21, wherein the physical agent is heat, light, pressure, magnetic fields, X-radiation, or non-thermal microwave radiation.

24. The method of claim 1, wherein the screening method is performed in a microarray format.

25. The method of claim 1, wherein the cell panel comprises at least one non-human mammalian cell.

26. The method of claim 1, wherein the cell panel comprises at least one human cell.
